SEQUENCE LISTING (16 sequences)

SEQ ID NO: 1 - DNA, 1728 bp, Homo Sapiens
atgcggccatggactggttcctggcgttggattatgctcattctttttgcctgggggaccttgctgttttatataggtggtcacttggtacgagataatgaccatcctgatcactctagccgagaactgtccaagattctggcaaagcttgaacgcttaaaacaacagaatgaagacttgaggcgaatggccgaatctctccggataccagaaggccctattgatcaggggccagctataggaagagtacgcgttttagaagagcagcttgttaaggccaaagaacagattgaaaattacaagaaacagaccagaaatggtctggggaaggatcatgaaatcctgaggaggaggattgaaaatggagctaaagagctctggtttttcctacagagtgaattgaagaaattaaagaacttagaaggaaatgaactccaaagacatgcagatgaatttcttttggatttaggacatcatgaaaggtctataatgacggatctatactacctcagtcagacagatggagcaggtgattggcgggaaaaagaggccaaagatctgacagaactggttcagcggagaataacatatcttcagaatcccaaggactgcagcaaagccaaaaagctggtgtgtaatatcaacaaaggctgtggctatggctgtcagctccatcatgtggtctactgcttcatgattgcatatggcacccagcgaacactcatcttggaatctcagaattggcgctatgctactggtggatgggagactgtatttaggcctgtaagtgagacatgcacagacagatctggcatctccactggacactggtcaggtgaagtgaaggacaaaaatgttcaagtggtcgagcttcccattgtagacagtcttcatccccgtcctccatatttacccttggctgtaccagaagacctcgcagatcgacttgtacgagtgcatggtgaccctgcagtgtggtgggtgtctcagtttgtcaaatacttgatccgcccacagccttggctagaaaaagaaatagaagaagccaccaagaagcttggcttcaaacatccagttattggagtccatgtcagacgcacagacaaagtgggaacagaagctgccttccatcccattgaagagtacatggtgcatgttgaagaacattttcagcttcttgcacgcagaatgcaagtggacaaaaaaagagtgtatttggccacagatgacccttctttattaaaggaggcaaaaacaaagtaccccaattatgaatttattagtgataactctatttcctggtcagctggactgcacaatcgatacacagaaaattcacttcgtggagtgatcctggatatacattttctctctcaggcagacttcctagtgtgtactttttcatcccaggtctgtcgagttgcttatgaaattatgcaaacactacatcctgatgcctctgcaaacttccattctttagatgacatctactattttgggggccagaatgcccacaatcaaattgccatttatgctcaccaaccccgaactgcagatgaaattcccatggaacctggagatatcattggtgtggctggaaatcattgggatggctattctaaaggtgtcaacaggaaattgggaaggacgggcctatatccctcctacaaagttcgagagaagatagaaacggtcaagtaccccacatatcctgaggctgagaaataa

SEQ ID NO: 2 - protein, 575 aa, Homo Sapiens
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 3 - DNA, 58 bp, Artificial Sequence
  Synthetic Polynucleotide
ccgggtctataatgacggatctatactcgagtatagatccgtcattatagactttttg

SEQ ID NO: 4 - DNA, 58 bp, Artificial Sequence
  Synthetic Polynucleotide
ccggccacagatgacccttctttatctcgagataaagaagggtcatctgtggtttttg

SEQ ID NO: 5 - DNA, 58 bp, Artificial Sequence
  Synthetic Polynucleotide
ccgggaactggttcagcggagaatactcgagtattctccgctgaaccagttctttttg

SEQ ID NO: 6 - DNA, 58 bp, Artificial Sequence
  Synthetic Polynucleotide
ccggctttagatgacatctactattctcgagaatagtagatgtcatctaaagtttttg

SEQ ID NO: 7 - DNA, 58 bp, Artificial Sequence
  Synthetic Polynucleotide
ccggtacccatgcacagtacaataactcgagttattgtactgtgcatgggtatttttg

SEQ ID NO: 8 - DNA, 728 bp, Artificial Sequence
  Synthetic Polynucleotide
gcgcgtcgacgatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaactggttcagcggagaatactcgagtattctccgctgaaccagttctttttggtcatgttcttaatcgatactagtgctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcccggccacagatgacccttctttatctcgagataaagaagggtcatctgtggtttttggaagtcgacgcgc

SEQ ID NO: 9 - DNA, 910 bp, Artificial Sequence
  Synthetic Polynucleotide
gcgcgtcgacgatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaactggttcagcggagaatactcgagtattctccgctgaaccagttctttttgcgacgttgtaaaacgacggccagtgaattcatatttgcatgtcgctatgtgttctgggaaatcaccataaacgtgaaatgtctttggatttgggaatcttataagttctgtatgagaccactcgccgggtctataatgacggatctatactcgagtatagatccgtcattatagactttttggtcatgttcttaatcgatactagtgctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcccggccacagatgacccttctttatctcgagataaagaagggtcatctgtggtttttggaagtcgacgcgc

SEQ ID NO: 10 - DNA, 492 bp, Artificial Sequence
  Synthetic Polynucleotide
ggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaactggttcagcggagaatactcgagtattctccgctgaaccagttctttttggaagtcgacgcgc

SEQ ID NO: 11 - DNA, 808 bp, Artificial Sequence
  Synthetic Polynucleotide
gcgcgtcgacgatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaactggttcagcggagaatactcgagtattctccgctgaaccagttctttttgaaggatctcaagaagatcctttgatcttttctacggggtctgacgctcagtggaacgaaaactcacgttaagggattttggtcatgttcttaatcgatactagtgctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcccggccacagatgacccttctttatctcgagataaagaagggtcatctgtggtttttggaagtcgacgcgc

SEQ ID NO: 12 - DNA, 1010 bp, Artificial Sequence
  Synthetic Polynucleotide
gcgcgtcgacgatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaactggttcagcggagaatactcgagtattctccgctgaaccagttctttttggatgtgctgcaaggcgattaagttgggtaacgccagggttttcccagtcacgacgttgtaaaacgacggccagtgaattcatatttgcatgtcgctatgtgttctgggaaatcaccataaacgtgaaatgtctttggatttgggaatcttataagttctgtatgagaccactcgccgggtctataatgacggatctatactcgagtatagatccgtcattatagactttttgctacggggtctgacgctcagtggaacgaaaactcacgttaagggattttggtcatgttcttaatcgatactagtgctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcccggccacagatgacccttctttatctcgagataaagaagggtcatctgtggtttttggaagtcgacgcgc

SEQ ID NO: 13 - DNA, 8547 bp, Artificial Sequence
  Synthetic Polynucleotide
gctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaacttccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcctagggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcagagccgccaccatgaagcacctctggtttttcctcctcctcgtcgcagcacctagatgggtcctcagtgaagtgcagctcctcgaacagtctggggtcgaagtgaagaaacccggcagctccgtcaaggtgtcctgcaaagcttctgggtacgcattcaccgactactatatccactgggtgcggcaggcacctaggcagggactggagtggatgggatggattaacccatacaatgggaagacaaaaagcgcccagaactttcagggaagggtgactatgaccagagacacaagtactgccaccgcttatatggacctctctagtctgacctccgaggatacagccatctactattgcgctagggatctgggctactgctctgacggcgtgtgctatgatggagccggcattccccctgacttcatcggcattcctgctgtgccaatgaatagcctcgatgtctggggaaggggagtcctggtgacagtctcaagcgcatcaactaagggaccaagcgtgtttccactggcaccttcctctcgcagtacttcagagagcaccgccgctctcggatgtctggtgaaagactacttcccagaacccgtgaccgtctcatggaactccggatctctgaccagcggcgtgcacacatttcctgccgtcctgcagagttcaggcctctacagcctgagctccgtggtcactgtgccatctagttcactggggacacagacttacgtgtgcaacgtcaatcataagccctctaatacaaaggtggataaaagggtcgaaatcaagacttgtggcgggggaagcaaaccacccacctgccctccatgtcctgcaccagagctgctcggaggaccatccgtgttcctgtttccccctaagcccaaagacaccctgatgatttctagaacacccgaagtgacttgcgtggtcgtggatgtcagccaggaagaccctgatgtgaagttcaactggtacgtgaatggggcagaggtccaccatgcccagactaaacccagggaaacccagtacaactccacatatagagtcgtgtctgtgctgaccgtcacacatcaggactggctgaacggcaaggaatacacctgcaaggtgagcaacaaggccctgcccgcccctatccagaaaacaatttccaaggataaagggcagccacgggagccccaggtgtatactctcccaccctcccgcgaggaactgaccaagaaccaggtgtctctcacatgtctggtcaaagggttctacccaagcgacatcgtcgtggagtgggaaagctccggacagcccgagaatacctataagaccacacctccagtgctggacagcgatggatcctacttcctctatagcaagctgacagtggataaatcccggtggcagcagggcaacgtgtttagttgctcagtcatgcacgaggccctgcacaatcattacacccagaagagcctctccctgtctcccggcaagcgcaaaaggagaagtggctcaggggctcctgtgaagcagacactcaactttgacctgctcaaactggcaggcgatgtggagagcaatccagggcccatgaagcatctgtggttctttctgctcctggtggcagcacctagatgggtgctctccgagctggtcatgacacagtctccactcagtctggctgtgactcctggagaaccagcaagcatctcctgtaggtctagtcagagtctcctggactcagaggatggaaacacctacctcgactggtatctgcagagacccggccagagccctcagctcctgatctacgaggtcagcaatcgggcatccggcgtgccagaccgcttctctggaagtggctcagacacagacttcaccctgaagatttccagggtggaggctgaagatgtgggagtctactattgcatgcagggcctcgagttccctctgacttttggaggcgggaccaaggtggaaatcaaaagagccgtggctgcaccatctgtcttcatttttccccctagtgaagaccaggtgaagtctggcaccgtcagtgtggtgtgcctcctgaacaatttctaccctcgcgaggcttctgtgaagtggaaagtggatggggtcctgaagactggaaactcacaggagagcgtgaccgaacaggacagtaaggataacacatactcactgtcaagcactctcaccctgtcctctaccgactaccagagccacaacgtgtatgcctgcgaagtcacccatcaggggctgtccagtccagtcaccaaatctttcaataggggcgagtgttgagcggccgcgaattcactagttccataaagtaggaaacactacatccataaagtaggaaacactacatccataaagtaggaaacactacatccataaagtaggaaacactacagctagccgcattattactcacggtacgacgcattattactcacggtacgacgcattattactcacggtacgacgcattattactcacggtacgacttaagaccggtggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacgatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaactggttcagcggagaatactcgagtattctccgctgaaccagttctttttggatgtgctgcaaggcgattaagttgggtaacgccagggttttcccagtcacgacgttgtaaaacgacggccagtgaattcatatttgcatgtcgctatgtgttctgggaaatcaccataaacgtgaaatgtctttggatttgggaatcttataagttctgtatgagaccactcgccgggtctataatgacggatctatactcgagtatagatccgtcattatagactttttgctacggggtctgacgctcagtggaacgaaaactcacgttaagggattttggtcatgttcttaatcgatactagtgctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcccggccacagatgacccttctttatctcgagataaagaagggtcatctgtggtttttggaagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggc

SEQ ID NO: 14 - DNA, 20 bp, Artificial Sequence
  Synthetic Polynucleotide
acgcgtactcttcctatagc

SEQ ID NO: 15 - DNA, 20 bp, Artificial Sequence
  Synthetic Polynucleotide
attgatcaggggccagctat

SEQ ID NO: 16 - DNA, 20 bp, Artificial Sequence
  Synthetic Polynucleotide
tactacctcagtcagacaga

(19) United States
(12) Patent Application Publication
(10) Pub. No.: US 2022/0002387 A1
(43) Pub. Date: Jan. 6, 2022

(54) Compositions and Production of Recombinant AAV Viral Vectors Capable of Glycoengineering In Vivo

(71) Applicant: UNIVERSITY OF MIAMI, Miami, FL (US)

(72) Inventors: James M. Termini, Miami, FL (US); Ronald Desrosiers, Miami, FL (US)

(21) Appl. No.: 17/291,884

(22) PCT Filed: Nov. 6, 2019
(86) PCT No.: PCT/US19/60142

§ 371 Date: May 6, 2021

Related U.S. Application Data
(60) Provisional application No. 62/756,233, filed on Nov. 6, 2018.

(51) Int. Cl. C07K 16/00; C12N 15/86; C12N 15/113; C12N 9/10

(52) U.S. Cl. CPC C07K 16/00; C12N 15/86; C12N 15/1137; C12N 9/1051; C07K 2317/41; C12N 2310/531; C12N 2750/14143; C07K 2317/52; C07K 2317/732; C12Y 204/01068

(57) ABSTRACT

The disclosure provides an expression vector (e.g., AAV vector) comprising a nucleic acid sequence encoding (1) the heavy and/or light chain of an antibody and (2) one or more shRNA sequences targeting fucosyltransferase-8 (FUT8).

### CROSS REFERENCE TO RELATED APPLICATION

This application claims the benefit of U.S. Provisional Patent Application No. 62/756,233, filed on Nov. 6, 2018, the entire contents of which is fully incorporated herein by reference.

### STATEMENT OF FEDERALLY SPONSORED RESEARCH

This invention was made with government support under grant number R01 AI098446, awarded by the National Institute of Allergy and Infectious Disease (NIAID). The government has certain rights in the invention.

### INCORPORATION BY REFERENCE OF TO MATERIALS SUBMITTED ELECTRONICALLY

This application contains, as a separate part of the disclosure, a Sequence Listing in computer readable form (Filename: 53652_Seqlisting.txt; Size: 26,417 bytes; Created: Nov. 6, 2019), which is incorporated by reference in its entirety.

### FIELD OF THE INVENTION

The present disclosure relates to a novel composition of matter and method of manufacture for recombinant AAV viral vectors capable of glycoengineering proteins in vivo.

### BACKGROUND

The constant domain of human IgG contains a single N-linked oligosaccharide at asparagine-297. The absence of fucose on the carbohydrate at this site can have a dramatic impact on antibody-dependent cellular cytotoxicity (ADCC) activity. Non-fucosylated antibody has been shown to increase ADCC activity from 10-100 times that of the fucosylated version. These findings have generated much interest in the therapeutic antibody field. Glycoengineered versions of several therapeutic antibodies for cancer are already in clinical trials. Current techniques of glycoengineering do not apply to antibody production methods using viral vectors such as adeno-associated viral vectors.

### SUMMARY OF THE INVENTION

The disclosure provides a method of glycoengineering a recombinant adeno-associated viral (AAV)-vector in vivo. In particular, the present disclosure relates to a novel composition of matter and method of manufacture for recombinant AAV viral vectors capable of being glycoengineered in vivo.

The disclosure provides an expression vector comprising a nucleic acid sequence encoding (1) the heavy and/or light chain of an antibody and (2) one or more shRNA sequences targeting fucosyltransferase-8 (FUT8).

The disclosure also provides a composition comprising (a) an expression vector comprising a nucleic acid sequence encoding a heavy chain of an antibody and (b) an expression vector comprising a nucleic acid sequence encoding a light chain of an antibody, where (a), (b), or (a) and (b) further comprise one or more shRNA sequences targeting fucosyltransferase-8 (FUT8).

The disclosure further provides a method of producing an antibody in vivo, the method comprising delivering to a subject (a) a nucleic acid comprising a nucleic acid sequence encoding a heavy chain of an antibody, (b) a nucleic acid comprising a nucleic acid sequence encoding a light chain of an antibody, and (c) an inhibitory RNA targeting fucosyltransferase-8 (FUT8).

### BRIEF DESCRIPTION OF THE FIGURES

FIG. 1: Generation of a FUT8 KO Cell Line. HEK293T cells were transfected with three gRNA targeting FUT8 and a CAS9/GFP expression plasmid. Four of the FUT8 KO clones isolated from cell sorting and a HEK293T wildtype control were transfected with an Ab 10-1074 expression plasmid. After an additional 4 days, supernatant was harvested and filtered to remove cell debris. IgG was purified by protein A column and 3 μg ran on 4-12% bris-tris gels in duplicate. After transfer, one membrane was stained with anti-IgG-HRP and the other was probed with AAL-HRP lectin to visualize the presence of al-6 fucose.

FIGS. 2A-2D: FUT8 shRNA and Cloning Strategy for Glycoengineering AAV (GE-AAV) Constructs. Five candidate shRNAs (shRNA 52[TRCN0000035952], shRNA 53[TRCN0000035953], shRNA 59[TRCN0000229959], shRNA 60 [TRCN0000229960], and shRNA 61[TRCN0000229961]) were selected for regions of FUT8 with >99% homology between human and rhesus macaque FUT8. FIG. 2A) Candidate shRNAs (in caps) were aligned to rhesus macaque FUT8 (lowercase) to demonstrate the homology. Only shRNA 61 had a one base pair difference when compared to the rhesus FUT8 sequence. FIG. 2B) HEK293T cells were transfected with pLKO.1 expression vector with each candidate shRNA. After 24 hours, cells were harvested and analyzed for FUT8 mRNA expression by real-time PCR. Data are presented as percentage knockdown compared to wildtype HEK-293T cells. FIG. 2C) Diagram depicting the design of the GE-AAV knockdown constructs. The poly A depicted is the poly A tail of the IgG being expressed by the AAV. U6, H1, and 7SK promoters were used to drive the expression of individual shRNAs. Spacer size was adjusted between constructs to maximize knockdown while maintaining maximal IgG expression. Constructs were designed using multiple pol III promoters each driving an individual short hairpin RNA (s)hRNA. Care was taken with the length of spacer sequences. FIG. 2D) Diagram depicting the cloning of the FUT8 knockdown constructs into the AAV vector. The construct was inserted downstream of the poly A tail and upstream of the 3′ ITR.

FIGS. 3A-3B: FUT8 knockdown validation by GE-AAV Constructs. HEK293T cells were transfected with plasmid DNA of the AAV vector plasmids containing the shRNA constructs outlined in FIG. 2. FIG. 3A) After 24 hours, cells were harvested and analyzed for FUT8 mRNA by real-time PCR. Data are presented as percentage knockdown compared to wildtype HEK-293T cells. FIG. 3B) AAL lectin western blot to detect fucose content of 4L6 antibody. HEK293T wildtype control were transfected with GE-AAV vectors expressing 4L6 antibody with or without a FUT8 shRNA construct. After 18 hours, cells were washed and transferred to serum free media for an additional 3 days. Media was aspirated and replaced with fresh serum free media to eliminate IgG produced before full knockdown of FUT8. On day 7, supernatant was harvested and filtered to remove cell debris. IgG was purified using a protein A column and 3 μg were analyzed on 4-12% bris-tris gels in duplicate. After transfer, one membrane was stained with anti-IgG-HRP and the other was probed with AAL-HRP lectin to visualize the presence of al-6 fucose. Fucose knockdown in lanes 2-5 was compared to 4L6 antibody produced in absence of shRNA (Lane 1).

FIG. 4: shRNA construct ADCC activity validation. HEK293T cells were transduced with lentivirus expressing constructs 2, 5, and 6. Lentiviruses also expressed a GFP tag along with a selectable puromycin resistance gene. 48 hours after transduction, puromycin was added to the cell media and allowed to select for positively transduced cells. For an additional 2 weeks, puromycin concentration was gradually increased to select a population of high expressing cells. 10-1074 IgG1 expressing plasmids were transfected into wildtype HEK293T cells, FUT8 knockout cell lines, and lenti-transduced HEK293T cell lines expressing constructs 2, 5, or 6. Ab 10-1074 was purified by protein A column and quantified. The dashed line indicates 50% RLU (relative light units) or 50% ADCC activity against HIV AD8-infected target cells. The loss of RLU indicates the loss of virus-infected cells during the 8-hour incubation period in the presence of a CD16⁺ NK cell line and a serial dilution of antibodies. The loss of RLU represents a high ADCC activity. Ab 10-1074 FUT8 was included as a positive control due to the complete lack of fucose on the purified IgG.

FIGS. 5A-5D: ADCC of common Ab 10-1074 Fc variants lacking fucose. Ab 10-1074 was produced in both HEK293T cells and FUT8 KO cell lines as wildtype IgG, LS mutant, LALA mutant, or a combination of LALA and LS mutations. Antibodies were purified by protein A column. FIG. 5A) 3 μg IgG was loaded onto a 4-12% bis-tris gel in duplicates. One was processed for Coomassie staining, the second was probed with AAL lectin to monitor the presence of α(1-6)fucose. FIG. 5B) SF162 gp140 trimer binding ELISA. Starting at an antibody concentration of 1 μg/ml followed by 3-fold serial dilutions, 10-1074 variants were incubated for ELISA measurement against SF162 gp140 trimer. High absorbance indicates high binding. FIG. 5C) Neutralization curve of AD8 with 10-1074 IgG1 starting at 1 μg/ml. The dashed line indicates 50% RLU (relative light units) representing 50% neutralization activity against the AD8 strain of HIV. Lowest RLU indicates highest neutralization. FIG. 5D) 10-1074 variants were tested for ADCC activity. The dashed line indicates 50% RLU (relative light units) or 50% ADCC activity against HIV AD8-infected target cells. ADCC was measured by the luciferase activity in HIV-infected cells after an 8-hour incubation in the presence of a human CD16⁺ NK cell line and a serial dilution of antibodies. The loss of RLU indicates the loss of virus-infected cells during the 8-hour incubation period and represents a high ADCC activity.

FIGS. 6A-6D: ADCC of Common 3BNC117 Fc variants lacking fucose. Ab 3BNC117 was made in both HEK293T cells and FUT8 KO cell lines as wildtype IgG, LS mutant, LALA mutant, or a combination of LALA and LS mutations. Antibodies were purified by protein A column. FIG. 6A) 3 μg IgG was loaded onto a 4-12% bis-tris gel in duplicates. One gel was processed for Coomassie staining, the second was transferred and probed with AAL lectin to monitor the present of α(1-6)fucose. FIG. 6B) SF162 gp140 trimer binding ELISA. Starting at an antibody concentration of 1 μg/ml followed by 3-fold serial dilutions, 3BNC117 variants were incubated for ELISA measurement against SF162 gp140 trimer. High absorbance indicates high binding. FIG. 6C) Neutralization curve of AD8 with 3BNC117 IgG1 starting at 1 μg/ml. The dashed line indicates 50% RLU (relative light units) representing 50% neutralization activity against the AD8 strain of HIV. Lowest RLU indicates highest neutralization. FIG. 6D) 3BNC117 variants were tested for ADCC activity. The dashed line indicates 50% RLU (relative light units) or 50% ADCC activity against HIV AD8-infected target cells. ADCC was measured by the luciferase activity in HIV-infected cells after an 8-hour incubation in the presence of a human CD16⁺ NK cell line and a serial dilution of antibodies. The loss of RLU indicates the loss of virus-infected cells during the 8-hour incubation period and represents a high ADCC activity.

FIGS. 7A-7D: Ab 10-1074 and Ab 3BNC117 ADCC enhancement by combining FUT8 removal with Fc mutation. Ab 10-1074 and Ab 3BNC117 were produced in HEK293T cells and FUT8 KO cells. FIGS. 7A & 7B) Antibodies were also made with the S239 mutations (S239D/I332F/A330L). FIGS. 7C & 7D) Asymmetric antibodies where different mutations are present on each heavy chain were also tested in both HEK293T and FUT8 KO cells. Variants tested were W117 (comprising a first heavy chain comprising mutations K392D/K409D/A330M/K334V and a second heavy chain comprising mutations E356K/D399K/L234Y/Y296W), W187 (comprising a first heavy chain comprising mutations K392D/K409D/S239D/A330M/K334V and a second heavy chain comprising mutations E356K/D399K/L234Y/K290Y/Y296W), W141 (comprising a first heavy chain comprising mutations K392D/K409D/A330M/K334V and a second heavy chain comprising mutations E356K/D399K/L234Y/K290Y/Y296W), W144 (comprising a first heavy chain comprising mutations K392D/K409D/A330F/K334V and a second heavy chain comprising mutations E356K/D399K/L234Y/K290Y/Y296W), and W125 (comprising a first heavy chain comprising mutations K392D/K409D/A330M/K334V and a second heavy chain comprising mutations W125B E356K/D399K/K290Y/Y296W). For asymmetric antibodies, equal amounts of plasmid for each heavy chain were cotransfected allowing the hybrid antibodies to be formed. Variants were tested for ADCC activity. The dashed line indicates 50% RLU (relative light units) or 50% ADCC activity against HIV AD8-infected target cells. ADCC was measured by the luciferase activity in HIV-infected cells after an 8-hour incubation in the presence of a human CD16+NK cell line and a serial dilution of antibodies. The loss of RLU indicates the loss of virus-infected cells during the 8-hour incubation period and represents a high ADCC activity. FIG. 7A) 10-1074 IgG1 variant ADCC assay. FIG. 7B) 3BNC117 IgG1 variant ADCC assay. FIG. 7C) 10-1074 asymmetric IgG1 variant ADCC assay. FIG. 7D) 3BNC117 asymmetric IgG1 variant ADCC assay.

### DETAILED DESCRIPTION

The present disclosure relates to a novel composition of matter and method of manufacture using recombinant AAV viral vectors capable of glycoengineering in vivo.

Despite the promising success of AAV-delivered IgG, there is room for optimization of the delivered antibodies. Therapeutic antibodies are commonly used for the treatment in cancer.³⁻⁶ To maximize the efficiency of cancer treatment, modification to the Fc structure and modification of glycan content of IgG increases antibody Fc binding and effector functions. Of these methods, glycoengineering is by far one of the most effective for modulating ADCC. IgG contains a single N-linked oligosaccharide at asn-297. This asn-297 contains an optional al-6 fucose residue on the first N-acetylglucosamine. A drastic increase in effector function is associated with the removal of the α1-6 fucose at asn-297, leading to enhancement of antibody-dependent cellular cytotoxicity (ADCC), a key mechanism of anti-cancer therapeutic antibodies. When anti-CD20 IgG1 (rituximab) was made in a non-fucosylated form, a 100-fold increase in B-cell depleting activity and higher affinity for FcγRIIIA binding was observed.¹¹ Much lower concentrations of antibody were necessary to achieve identical clinical efficacy.

Glycoengineering has enormous potential for treatment of HIV. High levels of ADCC have been associated with slowed progression, better viral control, and lower viral set points.¹⁶⁻¹⁸ Glycoengineering has also been successful in enhancing anti-HIV antibodies. When b12 was produced devoid of fucosylation, 10-fold higher viral inhibition was observed when compared to wild-type-b12.¹⁹ ADCC has also been suggested to be effective in the clearance of reactivated latent HIV-1 reservoirs. These findings suggest that glycoengineering may be an important avenue to pursue in the search for a functional cure for HIV.

The disclosure provides materials and methods for glycoengineering antibodies produced in vivo. In one aspect, the materials and methods employ inhibitory oligonucleotide that targets fucosyltransferase-8 (FUT8), preferably human FUT8 (and, optionally, rhesus FUT8). The fucosyltransferase-8 (FUT8) gene encodes α-(1,6)-fucosyltransferase, which catalyzes the transfer of fucose from GDP-fucose to N-linked type complex glycopeptides. The nucleotide sequence of human FUT8 is provided as SEQ ID NO: 1 and the amino acid sequence is provided as SEQ ID NO:2. Using one or more inhibitory oligonucleotide(s) to reduce expression (i.e. “knockdown”) FUT8 in connection with expression vectors encoding antibodies, antibodies are produced in vivo with altered glycosylation patterns resulting in enhanced ADCC activity.

In various embodiments, the disclosure provides an expression vector comprising a nucleic acid sequence encoding (1) a heavy and/or a light chain of an antibody and (2) one or more inhibitory oligonucleotide sequences (e.g., inhibitory RNA sequences, such as shRNA sequences) targeting fucosyltransferase-8 (FUT8). In various aspects, the expression vector encodes both the heavy chain and the light chain of an antibody. In various aspects, the expression vector encodes a heavy and/or a light chain of an antibody and is combined in a composition with inhibitory oligonucleotide (e.g., inhibitory RNA, such as shRNA), optionally on a separate expression vector, which targets FUT8.

In certain embodiments, the inhibitory oligonucleotide is an antisense oligonucleotide, an inhibitory RNA (including siRNA or RNAi, or shRNA), a DNA enzyme, a ribozyme (optionally a hammerhead ribozyme), or an aptamer. In one embodiment, the oligonucleotide is complementary to at least 10 bases of the nucleotide sequence of SEQ ID NO: 1.

The specific sequence utilized in design of inhibitory oligonucleotides may be any contiguous sequence of nucleotides contained within the expressed gene message of the target. Factors that govern a target site for the inhibitory oligonucleotide sequence include the length of the oligonucleotide, binding affinity, and accessibility of the target sequence. Sequences may be screened in vitro for potency of their inhibitory activity using any suitable method, including the methods described below. In general it is known that most regions of the RNA (5′ and 3′ untranslated regions, AUG initiation, coding, splice junctions and introns) can be targeted using antisense oligonucleotides. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference in its entirety.

Among inhibitory RNA, shRNA offers advantages in silencing longevity and delivery options. See, e.g., Hannon et al., Nature, 431:371-378 (2004) for review. Vectors that produce shRNAs, which are processed intracellularly into short duplex RNAs having siRNA-like properties have been reported (Brummelkamp et al., Science, 296: 550-553 (2000); Paddison et al., Genes Dev., 16: 948-958 (2002)). A hairpin can be organized in either a left-handed hairpin (i.e., 5′-antisense-loop-sense-3′) or a right-handed hairpin (i.e., 5′-sense-loop-antisense-3′). The RNA may also contain overhangs at either the 5′ or 3′ end of either the sense strand or the antisense strand, depending upon the organization of the hairpin. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Preferably, there is only one phosphate group on the 5′ end of the region that will form the antisense strand following Dicer processing. In one exemplary embodiment, a right-handed hairpin can include a 5′ end (i.e., the free 5′ end of the sense region) that does not have a 5′ phosphate group, or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′-OH functional group (e.g., the 5′-most nucleotide is a 5′-deoxy nucleotide). In cases where the hairpin is a left-handed hairpin, preferably the 5′ carbon position of the 5′-most nucleotide is phosphorylated.

Hairpins that have stem lengths longer than 26 base pairs can be processed by Dicer such that some portions are not part of the resulting siRNA that facilitates mRNA degradation. Accordingly, the first region, which may comprise sense nucleotides, and the second region, which may comprise antisense nucleotides, may also contain a stretch of nucleotides that are complementary (or at least substantially complementary to each other), but are or are not the same as or complementary to the target mRNA. While the stem of the shRNA can be composed of complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

Modified shRNAs can retain the modifications in the post-Dicer processed duplex. In exemplary embodiments, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin. Also, Dicer processing of hairpins with this configuration can retain the 5′ end of the sense strand intact, thus preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality.

shRNA may comprise sequences that were selected at random, or according to any rational design selection procedure. For example, rational design algorithms are described in International Patent Publication No. WO 2004/045543 and U.S. Patent Publication No. 20050255487, the disclosures of which are incorporated herein by reference in their entireties. Additionally, it may be desirable to select sequences in whole or in part based on average internal stability profiles (“AISPs”) or regional internal stability profiles (“RISPs”) that may facilitate access or processing by cellular machinery.

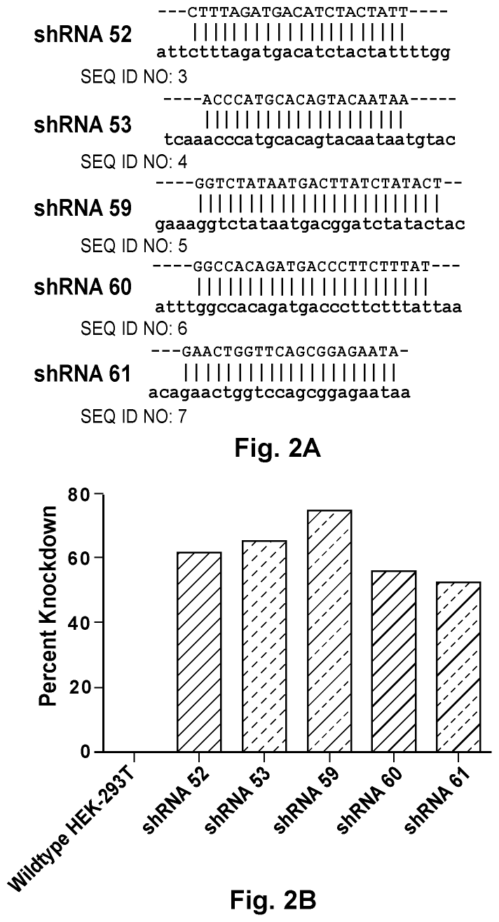

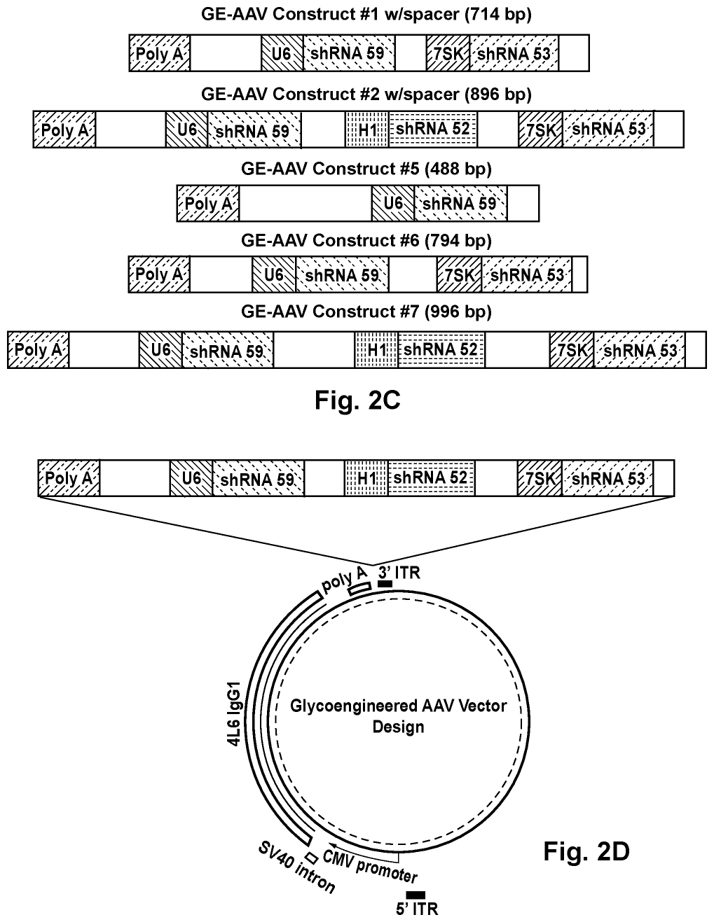

In various aspects of the disclosure, shRNA is used which comprises the nucleic acid sequence of any one of SEQ ID NOs: 3-7. In this regard, the disclosure provides an expression vector comprising one or more shRNA nucleic acid sequences which target FUT8. For example, the disclosure provides an expression vector comprising the nucleic acid sequence for shRNA59 (e.g., comprising the sequence of SEQ ID NO: 10); comprising the nucleic acid sequence for shRNA59 and the nucleic acid sequence encoding shRNA53 (e.g., comprising the sequence of SEQ ID NOs: 8 and 11); or comprising the nucleic acid sequence for shRNA59, the nucleic acid sequence encoding shRNA53, and the nucleic acid sequence encoding shRNA52 (e.g., comprising the sequence of SEQ ID NOs: 9, 12 or 13), each optionally operably linked to separate promoters. Exemplary constructs are illustrated in FIG. 2C.

A “vector” or “expression vector” is any type of genetic construct comprising a nucleic acid (DNA or RNA) for introduction into a host cell. In various embodiments, the expression vector is a viral vector, i.e., a virus particle comprising all or part of the viral genome, which can function as a nucleic acid delivery vehicle. Viral vectors comprising exogenous nucleic acid(s) encoding a gene product of interest also are referred to as recombinant viral vectors. As would be understood in the art, in some contexts, the term “viral vector” (and similar terms) may be used to refer to the vector genome in the absence of the viral capsid. Viral vectors for use in the context of the disclosure include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. Any of these viral vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989); Ausubel et al., Current Protocols in Molecular Biology, Greene Publishing Associates and John Wiley & Sons, New York, N.Y. (1994); Coen D. M, Molecular Genetics of Animal Viruses in Virology, 2nd Edition, B. N. Fields (editor), Raven Press, N.Y. (1990) and the references cited therein.

In any of the embodiments described herein, the expression vector is optionally an adeno-associated viral (AAV) vector. AAV is a DNA virus not known to cause human disease, making it a desirable gene therapy options. The AAV genome is comprised of two genes, rep and cap, flanked by inverted terminal repeats (ITRs), which contain recognition signals for DNA replication and viral packaging. AAV vectors used for administration of a therapeutic nucleic acid typically have a majority of the parental genome deleted, such that only the ITRs remain, although this is not required. As such, prolonged expression of therapeutic factors from AAV vectors can be useful in treating persistent and chronic diseases. The AAV vector is optionally based on AAV type 1, AAV type 2, AAV type 3 (including types 3A and 3B), AAV type 4, AAV type 5, AAV type 6, AAV type 7, AAV type 8, AAV type 9, AAV type 10, or AAV type 11. The genomic sequences of AAV, as well as the sequences of the ITRs, Rep proteins, and capsid subunits are known in the art. See, e.g., International Patent Publications Nos. WO 00/28061, WO 99/61601, WO 98/11244; as well as U.S. Pat. No. 6,156,303, Srivistava et al. (1983) J Virol. 45:555; Chiorini et al (1998) J Virol. 71:6823; Xiao et al (1999) J Virol. 73:3994; Shade et al (1986) J Virol. 58:921; and Gao et al (2002) Proc. Nat. Acad. Sci. USA 99:11854.

Expression vectors typically contain a variety of nucleic acid sequences necessary for the transcription and translation of an operably linked coding sequence. For example, expression vector can comprise origins of replication, polyadenylation signals, internal ribosome entry sites (IRES), promoters, enhancers, and the like. The vector of the disclosure preferably comprises a promoter operably linked to a coding sequence of interest (e.g., a nucleic acid sequence encoding a heavy chain and/or light chain of an antibody). “Operably linked” means that a control sequence, such as a promoter, is in a correct location and orientation in relation to another nucleic acid sequence to exert its effect (e.g., initiation of transcription) on the nucleic acid sequence. A promoter can be native or non-native to the nucleic acid sequence to which it is operably linked and native or non-native to a particular target cell type, and the promoter may be, in various aspects, a constitutive promoter, a tissue-specific promoter, or an inducible promoter (e.g., a promoter system comprising a Tet on/off element, a RU486-inducible promoter, or a rapamycin-inducible promoter). In various aspects, an expression vector is provided comprising a nucleic acid sequence encoding shRNA, which is operably linked to a Pol III promoter, such as III U6, 7SK, or H1 promoters. In certain embodiments the expression vector is pLKO.1.

Optionally, the virus coat or capsid (i.e., particle surface) is modified to adjust viral tropism. For example, the genome of one serotype of virus can be packaged into the capsid of a different serotype of virus to, e.g., evade the immune response. Alternatively (or in addition), components of the capsid can be modified to, e.g., expand the types of cells transduced by the resulting vector, avoid (in whole or in part) transduction of undesired cell types, or improve transduction efficiency of desired cell types. For example, transduction efficiency is generally determined by reference to a control (i.e., an unmodified, matched viral vector). Improvements in transduction efficiency can result in, e.g., at least about 25%, 50%, 60%, 70%, 80%, 85%, 90%, 95%, 100% improvement in transduction rate of a given cell type. If desired, the capsid can be modified such that it does not efficiently transduce non-target tissues, such as liver or germ cells (e.g., 50% or less, 30% or less, 20% or less, 10% or less, 5% or less of the level of transduction of desired target tissue(s)).

In various aspects, the expression vector comprises a nucleic acid sequence encoding a heavy chain and/or a light chain of an antibody. In various aspects, the expression vector encodes both a heavy chain and a light chain. The disclosure is not dependent on a particular antibody or encoding nucleic acid sequence. Optionally, the heavy chain comprises one or more mutations in the Fc region which enhances antibody-dependent cell cytotoxicity. For example, in various aspects, the heavy chain comprises one or more mutations selected from an LS mutation (M428L/N434S), a LALA mutation (L234A, L235A), a S239 (DFL) mutation (S239D/I332F/A330L), a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W), K392D/K409D/A330M/K334V, and/or E356K/D399K/K290Y/Y296W. In various embodiments, expression vectors encoding different heavy chains (i.e., a first heavy chain and a second heavy chain) are used, wherein the first heavy chain and the second heavy chain comprise different mutations (i.e., asymmetric antibodies where different mutations are present on each heavy chain). For example, in various aspects, the heavy chains comprise one or more mutations selected from W117 (comprising a first heavy chain comprising mutations K392D/K409D/A330M/K334V and a second heavy chain comprising mutations E356K/D399K/L234Y/Y296W), W187 (comprising a first heavy chain comprising mutations K392D/K409D/S239D/A330M/K334V and a second heavy chain comprising mutations E356K/D399K/L234Y/K290Y/Y296W), W141 (comprising a first heavy chain comprising mutations K392D/K409D/A330M/K334V and a second heavy chain comprising mutations E356K/D399K/L234Y/K290Y/Y296W), W144 (comprising a first heavy chain comprising mutations K392D/K409D/A330F/K334V and a second heavy chain comprising mutations E356K/D399K/L234Y/K290Y/Y296W), and W125 (comprising a first heavy chain comprising mutations K392D/K409D/A330M/K334V and second heavy chain comprising mutations W125B E356K/D399K/K290Y/Y296W). As demonstrated in the Examples, combination of Fc mutations with glycoengineering as described herein provides a surprising enhancement of ADCC activity.

The disclosure provides a composition comprising (a) an expression vector comprising a nucleic acid sequence encoding a heavy chain of an antibody and (b) an expression vector comprising a nucleic acid sequence encoding a light chain of an antibody, wherein (a), (b), or (a) and (b) further comprises one or more shRNA sequences targeting fucosyltransferase-8 (FUT8). Alternatively, the disclosure provides a composition comprising (a) an expression vector comprising a nucleic acid sequence encoding a heavy chain of an antibody, (b) an expression vector comprising a nucleic acid sequence encoding a light chain of an antibody, and (c) an expression vector comprising one or more shRNA sequences targeting fucosyltransferase-8 (FUT8) (such as, for example, the expression vectors described herein and illustrated in FIG. 2C). In various aspects, the expression vectors are adeno-associated viral (AAV) vectors. Optionally, the antibody heavy chain comprises one or more mutations in the Fc region which enhances antibody-dependent cell cytotoxicity, such as an LS mutation (M428L/N434S), a LALA mutation (L234A, L235A), a S239 (DFL) mutation (S239D/I332F/A330L), a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W), K392D/K409D/A330M/K334V, and/or E356K/D399K/K290Y/Y296W. In various embodiments, expression vectors encoding different heavy chains (i.e., a first heavy chain and a second heavy chain) are used, wherein the first heavy chain and second heavy chain comprise different mutations on each heavy chain (i.e., asymmetric antibodies where different mutations are present on each heavy chain). In various aspects the mutations on the first or second heavy chain may be interchanged. For example, in various aspects, the heavy chains comprise one or more mutations selected from W117 (comprising a first heavy chain comprising mutations K392D/K409D/A330M/K334V and a second heavy chain comprising mutations E356K/D399K/L234Y/Y296W), W187 (comprising a first heavy chain comprising mutations K392D/K409D/S239D/A330M/K334V and a second heavy chain comprising mutations E356K/D399K/L234Y/K290Y/Y296W), W141 (comprising a first heavy chain comprising mutations K392D/K409D/A330M/K334V and a second heavy chain comprising mutations E356K/D399K/L234Y/K290Y/Y296W), W144 (comprising a first heavy chain comprising mutations K392D/K409D/A330F/K334V and a second heavy chain comprising mutations E356K/D399K/L234Y/K290Y/Y296W), and W125 (comprising a first heavy chain comprising mutations K392D/K409D/A330M/K334V and second heavy chain comprising mutations W125B E356K/D399K/K290Y/Y296W).

The disclosure further provides a method of producing an antibody in vivo, the method comprising delivering to a subject an expression vector comprising a nucleic acid sequence encoding (1) a heavy and/or a light chain of an antibody and (2) one or more inhibitory oligonucleotide sequences (e.g., inhibitory RNA sequences, such as shRNA sequences) targeting fucosyltransferase-8 (FUT8). In various aspects, the expression vector encodes both the heavy chain and the light chain of an antibody.

Additionally, the disclosure provides a method of producing an antibody in vivo, the method comprising delivering to a subject a composition comprising (a) an expression vector comprising a nucleic acid sequence encoding a heavy chain of an antibody and (b) an expression vector comprising a nucleic acid sequence encoding a light chain of an antibody, wherein (a), (b), or (a) and (b) further comprises one or more inhibitory oligonucleotide (e.g., shRNA sequences) targeting fucosyltransferase-8 (FUT8). Alternatively, the disclosure provides a composition comprising (a) an expression vector comprising a nucleic acid sequence encoding a heavy chain of an antibody, (b) an expression vector comprising a nucleic acid sequence encoding a light chain of an antibody, and (c) an expression vector comprising one or more inhibitory oligonucleotide (e.g., shRNA sequences) targeting fucosyltransferase-8 (FUT8) (such as, for example, the expression vectors described herein and illustrated in FIG. 2C).

In various aspects, the disclosure provides a method of producing an antibody in vivo, the method comprising delivering to a subject (a) a nucleic acid comprising a nucleic acid sequence encoding a heavy chain of an antibody, (b) a nucleic acid comprising a nucleic acid sequence encoding a light chain of an antibody, and (c) an inhibitory oligonucleotide targeting fucosyltransferase-8 (FUT8). The nucleic acids of (a), (b), and (c) are optionally independently present on the same or different expression vectors (i.e., (a) and (b) may be on the same vector; (a) and (c) may be on the same vector; (b) and (c) may be on the same vector; (a), (b), and (c) may each be on different vectors, etc.). In various embodiments, the expression vectors are AAV vectors. Further, as described above, the heavy chain(s) optionally comprise one or more mutations in the Fc region which enhances antibody-dependent cell cytotoxicity, such as an LS mutation (M428L/N434S), a LALA mutation (L234A, L235A), and/or an S239 (DFL) mutation (S239D/I332F/A330L), a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W), K392D/K409D/A330M/K334V, and/or E356K/D399K/K290Y/Y296W. In various embodiments expression vectors encoding different heavy chains are used, wherein there is a first heavy chain and a second heavy chain comprising different mutations on each heavy chain (i.e., asymmetric antibodies where different mutations are present on each heavy chain of the antibody). For example, in various aspects, the heavy chains comprise one or more mutations selected from W117 (comprising a first heavy chain comprising mutations K392D/K409D/A330M/K334V and a second heavy chain comprising mutations E356K/D399K/L234Y/Y296W), W187 (comprising a first heavy chain comprising mutations K392D/K409D/S239D/A330M/K334V and a second heavy chain comprising mutations E356K/D399K/L234Y/K290Y/Y296W), W141 (comprising a first heavy chain comprising mutations K392D/K409D/A330M/K334V and a second heavy chain comprising mutations E356K/D399K/L234Y/K290Y/Y296W), W144 (comprising a first heavy chain comprising mutations K392D/K409D/A330F/K334V and a second heavy chain comprising mutations E356K/D399K/L234Y/K290Y/Y296W), and W125 (comprising a first heavy chain comprising mutations K392D/K409D/A330M/K334V and second heavy chain comprising mutations W125B E356K/D399K/K290Y/Y296W). Further, the inhibitory oligonucleotide is optionally shRNA, such as shRNA comprising the nucleic acid sequence of any one of SEQ ID NOs:3-7. In this regard, the method optionally comprises administering multiple shRNAs comprising different sequences selected from SEQ ID NOs: 3-7, which may be present on the same or different expression vectors.

The “subject” can be any mammal, such as a human. Contemplated mammalian subjects include, but are not limited to, animals of agricultural importance, such as bovine, equine, and porcine animals; animals serving as domestic pets, including canines and felines; and animals typically used in research, including rodents and primates.

In various aspects, the expression vector is provided in a composition (e.g., a pharmaceutical composition) comprising a physiologically-acceptable (i.e., pharmacologically-acceptable) carrier, buffer, excipient, or diluent. Any suitable physiologically-acceptable (e.g., pharmaceutically acceptable) carrier can be used within the context of the disclosure, and such carriers are well known in the art. The choice of carrier will be determined, in part, by the particular site to which the composition is to be administered and the particular method used to administer the composition. The composition also can comprise agents, which facilitate uptake of the expression vector into host cells. Suitable composition formulations include aqueous and non-aqueous solutions, isotonic sterile solutions, which can contain anti-oxidants, buffers, bacteriostats, and solutes that render the formulation isotonic with the blood, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. A composition comprising any one or more of the expression vectors described herein is, in one aspect, placed within containers, along with packaging material that provides instructions regarding the use of the composition. Generally, such instructions include a tangible expression describing the reagent concentration, as well as, in certain embodiments, relative amounts of excipient ingredients or diluents (e.g., water, saline or PBS) that may be necessary to reconstitute the composition.

The expression vector(s) (e.g., viral particle(s)) is administered in an amount and at a location sufficient to produce glycoengineered antibodies and, in various embodiments, provide some improvement or benefit to the subject. Depending on the circumstances, a composition comprising the expression vector(s) is applied or instilled into body cavities, applied directly to target tissue, and/or introduced into circulation. For example, in various circumstances, it will be desirable to deliver the composition comprising the expression vector by intravenous, intraperitoneal, intramuscular, or subcutaneous means.

A particular administration regimen for a particular subject will depend, in part, upon the amount of vector administered, the route of administration, and the cause and extent of any side effects. The amount administered to a subject (e.g., a mammal, such as a human) in accordance with the disclosure should be sufficient to produce antibodies at a desired over a reasonable time frame. Exemplary doses of viral particles in genomic equivalent titers of 10⁴-10¹⁵ transducing units (e.g., 10⁷-10¹² transducing units), or at least about 10⁵, 10⁶, 10⁷, 10⁸, 10⁹, 10¹⁰, 10¹¹, 10¹², 10¹³, 10¹⁴, or 10¹⁵ transducing units or more (e.g., at least about 10⁷, 10⁸, 10⁹, 10¹⁰, 10¹¹, 10¹², 10¹³, or 10¹⁴ transducing units, such as about 10¹⁰ or 10¹² transducing units).

As described herein, shRNA targeting fucosyltransferase-8 (FUT8), the glycosyltransferase responsible for fucosylation at asn-297 on IgG, were designed and tested. shRNA clones that exhibit sufficient knock-down by real-time PCR were cloned into the same AAV vector used to deliver HIV-specific broadly neutralizing antibodies. Antibody produced by our glycoengineered-AAV (GE-AAV) vectors was purified and analyzed for fucose content, neutralization, trimer binding and ADCC.

Conclusions: 1) AAV vectors can be engineered to efficiently express shRNA; 2) A spacer length of 75 b.p. or greater is preferred (but not required) to efficiently express all shRNA included; 3) Antibody produced by the GE-AAV constructs have only low levels of detectable al-6 fucose; 4) Although no difference was observed in neutralization or trimer binding, antibodies produced by the GE-AAV constructs have 10-100 fold higher ADCC activity depending on the antibody tested; 5) Can be combined with other forms of glycoengineering and Fc mutations to further enhance ADCC; and 6) Therapeutic benefit will be tested in macaques chronically infected with SHIV-AD8.

While preferred embodiments of the invention have been illustrated, it will be obvious to those skilled in the art that various modifications and changes may be made thereto without departing from the spirit and scope of the invention as hereinafter defined in the appended claims.

Additional aspects and details of the disclosure will be apparent from the following examples, which are intended to be illustrative rather than limiting.

### EXAMPLES

### General Methods

gRNA Generation. Guide RNAs (gRNAs) (SantaCruz Biotechnology) were generated to FUT8 (Gene ID: 2530). Target sequences were determined using the GeCKO v2 human library. Three gRNAs to FUT8 were used, targeting both strands of DNA, to ensure full knockout (KO) of the gene of interest. gRNAs were cloned into an expression vector with a GFP tag to allow for single cell GFP sort.

FUT8 CRISPR/Cas9 KO gRNAs:

(SEQ ID NO: 14)
1: ACGCGTACTCTTCCTATAGC
(SEQ ID NO: 15)
2: ATTGATCAGGGGCCAGCTAT
(SEQ ID NO: 16)
3: TACTACCTCAGTCAGACAGA

Cell Line Generation. HEK293T cells (ATCC) were transfected with three gRNAs for human FUT8 using JetPrime transfection reagent (Polyplus-Transfection). Cells were examined by GFP fluorescent microscopy at 24 hours post-transfection using a Zeiss Axio Observer A1 Microscope to gauge sufficient levels of expression necessary for downstream flow cytometric analysis and cell sorting. Following microscopy, cells were harvested, washed in PBS, and resuspended in DMEM with 1 mM EDTA to prevent the formation of cell aggregates. Cells were sorted on a 5 laser 17-color BD FACS SORP Aria-Hu with an Automatic Cell Deposition Unit (ACDU). The top 20% GFP-expressing cells were individually sorted into a 96-well plate. FSC-W by FSC-A and SSC-W by SSC-A were used to reduce the rate of duplets. Four hours post-sort, cells were inspected to ensure that all wells contained only one cell. Any wells that contained duplets were excluded from further processing. Once clones reached confluency in a 6-well plate, cells were lysed in RIPA buffer (Life Technologies) and used for western blot analysis.

Western Blots to Confirm FUT8 CRISPR Knockout. Four FUT8 KO clones were selected for further screening. HEK293T wildtype control and four FUT8 KO clones were transfected with a 10-1074 monoclonal antibody expression plasmid using JetPrime transfection reagent (Polyplus-Transfection). After 18 hours, cells were washed with and transferred to BIO-MPM-1 serum free media (Biological Industries). After an additional 4 days, supernatant was harvested and filtered through a 0.44 μm aPES filter (Thermo Scientific) to remove cell debris. IgG was purified using HiTrap protein A column (GE Healthcare) and 3 μg ran on 4-12% bris-tris gels (Life Technologies) in duplicate. 10-1074 antibody produced in wild-type HEK293T cells was loaded in the first lane as a control. Protein was transferred to a PVDF membrane using the iBlot Dry Blotting System (Life Technologies). After transfer, one membrane was probed with anti-human-IgG-HRP (SouthernBiotech) using the iBind western system (Life Technologies) and the other blot was probed with AAL-HRP lectin (BioWorld) to visualize the presence of al-6 fucose. Membranes were developed using the SuperSignal Pico Substrate (ThermoFisher) and images captured on an ImageQuant LAS 4000 mini Luminescent Image Analyzer (GE Healthcare).

FUT8 shRNA design. Five candidate shRNAs were selected to regions of human FUT8 with near identical homology between human and rhesus macaque FUT8. Candidate shRNAs were aligned to rhesus macaque FUT8 to demonstrate the homology using Serial Cloner 2-6-1. Candidate shRNA were cloned into the pLKO.1 expression vector to allow for transfection experiments.

FUT8 Realtime PCR. Wildtype HEK293T cells were transfected with pLKO.1 expression vector with each candidate shRNA, GE-AAV vector plasmids, and GFP expression vector where indicated using JetPrime transfection reagent (Polyplus-Transfection). After 24 hours, cells were harvested and washed with PBS. FUT8 shRNA expression was analyzed by real-time PCR using the TaqMan Gene Expression Assay HS00189535_ml (Life Technologies) and the Cells-to-CT 1-Step TaqMan kit (Life Technologies) according to manufacturer's specified protocol. Data are presented as percentage knockdown compared to wildtype HEK-293T cells.

GE-AAV cloning. Coding sequences for 4L6 and 10-1074 antibodies, were cloned into a single-stranded AAV (ssAAV) vector as previously described (Fuchs et al., PLoS One. 11(6):e0158009 (2016)) using a bicistronic expression cassette containing a F2A peptide and a furin peptide. All antibody sequences were codon-optimized and synthesized by Genscript.

shRNA constructs were constructed to include one, two, or three shRNA targeting various regions of FUT8 and all under the control of individual Pol III promoters. Pol III U6, 7SK, and H1 promoters were used. The strongest promoters were used to drive expression of the shRNA that exhibited the highest levels of knockdown by real-time PCR. shRNA constructs were cloned into the ssAAV vectors containing 4L6 and 10-1074 antibody sequences. shRNA constructs were inserted downstream of the poly A tail and upstream of the 3′ ITR. All GE-AAV vectors were tested for levels of knockdown by real-time PCR as described herein.

Lectin Western Blots to Confirm FUT8 Knockdown. GE-AAV vectors expressing 4L6 antibody and FUT8 shRNA knockdown constructs were transiently transfected into HEK293T cells using JetPrime transfection reagent (Polyplus-Transfection). After 18 hours, cells were washed and media was replaced with BIO-MPM-1 serum free media (Biological Industries). After an additional 4 days, supernatant was harvested and filtered through a 0.44 μm aPES filer (Thermo Scientific) to remove cell debris. IgG was purified using HiTrap protein A column (GE Healthcare) and 3 μg ran on 4-12% bris-tris gels (Life Technologies) in duplicate. 4L6 antibody produced in wild-type HEK293T cells was loaded in the first lane as a control. Protein was transferred to a PVDF membrane using the iBlot Dry Blotting System (Life Technologies). After transfer, one membrane was probed with anti-human-IgG-HRP (SouthernBiotech) using the iBind western system (Life Technologies) and the other blot was probed with AAL-HRP lectin (BioWorld) to visualize the presence of al-6 fucose. Lectin blots were blocked, probed, and washed using RIPA buffer (Life Technologies). Membranes were developed using the SuperSignal Pico Substrate (ThermoFisher) and images captured on an ImageQuant LAS 4000 mini Luminescent Image Analyzer (GE Healthcare).

shRNA Knockdown Cell Lines. shRNA constructs 2, 5, and 6 were cloned into the pGFP-C-shLenti lentiviral vector. Lentivirus was packaged using the Lenti-vpak packaging kit (Origene) according to manufacturer's specified protocol. HEK293T cells were plated in a 6 well plate the day before transduction at a density in which cells would reach ˜70% confluence on the day of transduction. 1 ml of harvested viral supernatant was incubated with HEK293T cells for 48 hours before adding 1 μg/ml puromycin (Life Technologies). Puromycin dosage was escalated to 2 μg/ml after week one and 4 μg/ml after week 2 to select for well transduced cells. shRNA construct expression was monitored by flow cytometry for GFP expression in comparison to HEK293T wildtype control cells.

gp140 ELISA. 10-1074 and 3BNC117 variants were tested for their ability to bind SF162 gp140 trimer (NIH AIDS Reagent Program) by ELISA. High binding ELISA plates were coated with recombinant SF162 gp140 overnight at 4° C. in PBS. Plates were washed using PBS-Tween20 (Sigma-Aldrich) and subsequently blocked with 5% nonfat dry milk in PBS (Bio-Rad). 10-1074 and 3BNC117 variants were serially diluted 1:3 in blocking buffer and added to the test plate. After 1 h of incubation at 37° C. the plates were washed again and an HRP-conjugated goat anti-human IgG H+L (SouthernBiotech) was then added for detection. The reaction was stopped after 1 h at 37° C. and plates were washed 10 times. Subsequently, TMB substrate and stop solutions (SouthernBiotech) were added and Absorbance at 450 nm was measured in a microplate reader (PerkinElmer).

Viral Neutralization Assay. Neutralization assays against HIV-1 AD8 were performed in TZM-bl cells as previously described (Alpert et al., Journal of virology 86, 12039-12052 (2012)), using 2 ng HIV-1 p24 per well. 5,000 TZM-bl cells per well were plated in flat-bottom 96-well cellbind plates the day before neutralization assay. Antibody dilutions and viruses were incubated for 1 h at 37° C. before being combined with the TZM-bl reporter cells. Luciferase activity in TZM-bl cells was measured after 3 days using BriteLite Plus luciferase substrate (Perkin Elmer). The antibody titers required to neutralize 50% of the viral infection were calculated.

ADCC Assay. ADCC activity was measured by a previously established assay to quantify NK cell activity towards virus-infected target cells expressing luciferase as previously described (Alpert et al., Journal of virology 86, 12039-12052 (2012)) with slight modifications outlined below. Infection was carried out by spinoculation in round-bottom 12×75 mm tubes using 200 ng p24 HIV-1AD8. Virus and target cells were centrifuged for 2 h at 1,200×g at 25° C.

ADCC assays were performed in round-bottom, 96-well plates, with each well containing 10⁴ target cells and 10⁵ effector cells in 200 μl final volume. Effector cells were combined with washed target cells immediately before addition to assay plates. Four-fold serial dilutions of antibodies were performed in triplicate. Once targets, effectors, and serially diluted antibody were combined, assay plates were incubated for 8 h at 37° C. After an 8 hour incubation, plates were spun down and 100 μl media removed from the top. 100 μl of BriteLite Plus (Perkin Elmer) was added to each well and mixed by pipetting. 150 μl of the mixture was transferred to a white 96-well plate. Luciferase activity was read using a Wallac Victor plate reader (Perkin Elmer).

AAV Production. Production of rAAVs was conducted as described previously (Mueller et al., Protoc. Microbiol., Chapter 14 (2012) Unit 14D.1). HEK-293 cells were transfected with a rAAV vector plasmid and two helper plasmids to allow generation of infectious AAV particles. After harvesting transfected cells and cell culture supernatant, rAAV was purified by three sequential CsCl centrifugation steps. Vector genome number was assessed by Real-Time PCR, and the purity of the preparation was verified by electron microscopy and silver-stained SDS-PAGE.

AAV In Vitro Transduction. HEK293T cells were seeded in R10 media in 6-well cellbind plates (Corning) 1 day prior to transduction. On the day of AAV transduction, cells reached a confluency of −50-70% and were infected with a total of 2×10⁴ rAAV particles per cell. Cells were transduced with AAV expressing 3BNC117 antibody with or without shRNA targeting FUT8. Cell culture medium was changed 24 h after transduction to fresh R10. After an additional 3 days, media was changed again to BIO-MPM-1 serum free media (Biological Industries). 500 μl of supernatant was harvested and replaced with fresh serum-free media every 24 hours for an additional 4 days. Supernatant was clarified by centrifugation at 16,000 RCF and 4° C. for 10 min. Concentration of secreted 3BNC117 IgG1 in cell culture supernatant was measured by Protein A/anti-rhesus IgG ELISA using purified rhesus IgG as standard as previously described (Fuchs et al., PLoS One. 11(6):e0158009 (2016)). Equal amounts of 3BNC117 antibody were analyzed for al-6 fucose by lectin western blot as described herein.

### Example 1: Generation of FUT8 Glycosylation Deficient Cell Lines and Validation

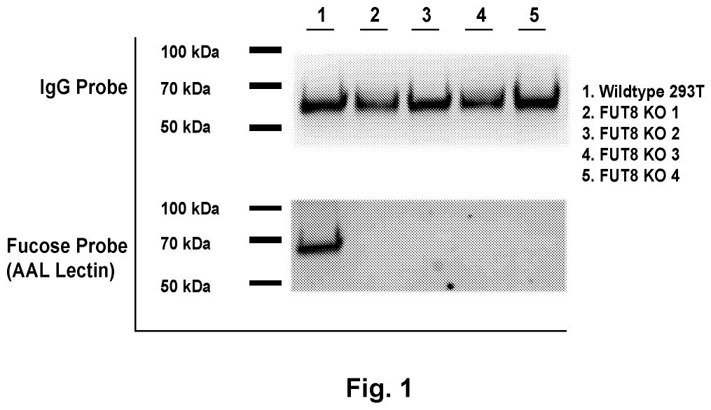

DNA sequences encoding guide RNAs (gRNAs) that target the human FUT8 gene were cloned into an expression vector containing a green fluorescent protein (GFP) tag. HEK293T cells were transiently transfected with three gRNA expression vectors all targeting FUT8. Cells were examined by GFP fluorescent microscopy at 24 hours post-transfection to gauge sufficient levels of expression necessary for downstream flow cytometric analysis and cell sorting. Cells were harvested and sorted using a FACS Aria II cell sorter with a 96-well plate adapter. Tight gating on forward scatter width (FSC-W) by forward scatter area (FSC-A) and side scatter width (SSC-W) by side scatter area (SSC-A) was used to reduce the frequency of duplets (FIG. 1A). Cells within the top 20% of GFP-expression were individually sorted, depositing an individual cell per well of a 96-well plate. Five hours post sort, wells were examined via confocal microscopy to ensure no well received more than 1 cell. Cells were allowed to grow to confluence before confirming that all copies of the target gene were disrupted.

Four HEK293T-FUT8 knockout clones (FUT8 KO) were analyzed for their ability to fucosylate human IgG. Expression vectors encoding the human anti-HIV mAb 10-1074 were transiently transfected into FUT8 KO clones as well as a HEK293T parental cell line. Five days post transfection, secreted 10-1074 was affinity purified using protein A columns. Purified antibodies were analyzed by western blot using an IgG probe to ensure equal amounts of protein were loaded in each lane and by lectin western blot using an AAL-HRP lectin to detect al-6 fucose present on the IgG (FIG. 1B). AAL lectin is known to bind specifically to al-6 fucose, which can only be added to a growing N-glycan chain by FUT8. Therefore, lack of al-6 fucose would suggest lack of FUT8 enzymatic activity.

Although equal amounts of IgG were loaded in each lane, there was no detectable al-6 fucose present on IgG produced in the four FUT8 KO cell lines (FIG. 1B).

### Example 2: FUT8 shRNA Design and Selection

Due to the high similarity between human and rhesus fucosyltransferase 8 genes (FUT8), five shRNAs were designed that are capable of targeting both human and rhesus FUT8. By selecting shRNA that can target both human and rhesus FUT8, GE-AAV vectors can be used in both in vitro work with human cell lines and used for macaque animal experiments. Candidate human shRNAs were aligned to rhesus macaque FUT8 to demonstrate the homology (FIG. 2A). Only shRNA 61 had a one base-pair difference when compared to the rhesus FUT8 sequence.

Candidate shRNAs were cloned into the pLKO.1 expression vector under the control of a U6 promoter. Expression vectors were transiently transfected into HEK293T cells and levels of FUT8 mRNA were measured by real-time PCR 24 hours after transfection. Although all clones exhibited high levels of knockdown, shRNAs 52, 53, and 59 mediated the highest levels of knockdown (FIG. 2B). All three clones exhibited greater than 60% knockdown, with clone 59 achieving the highest knockdown approaching 80%. These three shRNAs were chosen for the development of the GE-AAV vectors.

### Example 3: Design and Development of GE-AAV Vectors

GE-AAV constructs were designed not to exceed 1000 base pairs due to packaging limitation of the ssAAV vector. Constructs with varying spacer lengths and number of shRNA were tested (FIG. 2C). All shRNAs were designed to have independent Pol III promoters. U6, H1, and 7SK promoters were used to drive the expression of the individual shRNA. Care was taken to use the strongest promoter to drive the shRNA that exhibited the highest levels of FUT8 knockdown. shRNA constructs were cloned in the ssAAV vector downstream of the IgG Poly A tail and upstream of the 3′ ITR using an existing SalI restriction site and screening for proper orientation in the final constructions (FIG. 2D).

Spacer length was identified as an optimizable variable to the construct design. Early versions of construct #1 and #2 were found to reduce antibody production due to short spacers between the end of the IgG Poly A tail and the beginning of the U6 promoter. Once this spacer length was increased, expression was restored to normal levels. Similarly, if the individual shRNA and the start of the next Pol III promoter were in close proximity, a decrease in FUT8 knockdown was observed. Spacer length was optimized to allow for maximal FUT8 knockdown without inhibiting IgG expression. Spacer length of about 75 base pairs (b.p.) is preferred, but not required. The five constructs presented here are a result of this optimization.

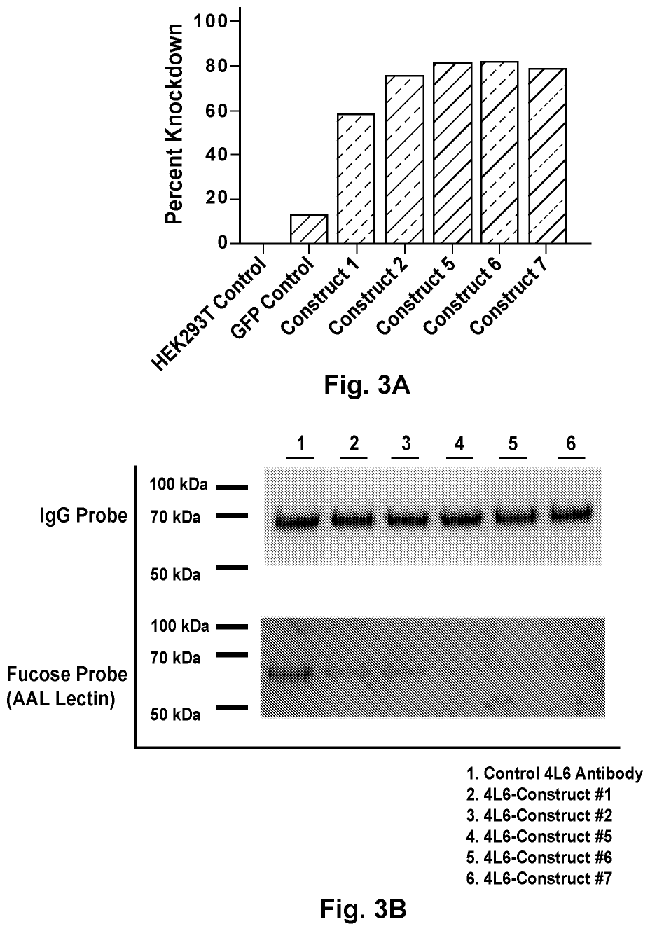

Validation of GE-AAV Vectors by rt-PCR. AAV plasmid DNA encoding 4L6 IgG and the various FUT8 shRNA constructs was transiently transfected into HEK293T cells. After 24 hours, cells were harvested and washed in PBS. Realtime PCR was performed on the transfected cells in order to measure levels of human FUT8 mRNA. Percent knockdown was calculated compared to FUT8 mRNA levels present in non-transfected HEK293T control cells. Constructs 5, 6, and 7 (SEQ ID NOs: 11-13) demonstrated relative knockdown of approximately 80% (FIG. 3A). Surprisingly, construct 5, while only containing one shRNA, demonstrated similar levels of knockdown to construct 7 which contains three different shRNAs targeting FUT8.

As previously mentioned, spacer length between shRNA clones can impact the expression levels of the individual shRNA. Although construct 1 (SEQ ID NO: 8) and construct 6 (SEQ ID NO:11) contain the exact same shRNAs and promotors, there is a 22% difference in knockdown. The only difference between these two clones is the larger spacer length between shRNA 59 and the 7SK promoter. Similarly, construct 2 and construct 7 also contain the same shRNAs and promoters yet construct 7 demonstrated a 5% enhancement in knockdown due solely to large spacer sequences between each shRNA and the downstream promoter.

Validation of GE-AAV Vectors by Lectin Western Blot. AAV plasmid DNA encoding 4L6 IgG and the various FUT8 shRNA constructs was transiently transfected into HEK293T cells. After 18 hours, media was replaced with R10 complete media. After an additional 3 days, cells were washed and media was replaced with serum free media. 4 days post media change, supernatants were harvested and filtered. 4L6 IgG present in the supernatant was purified and 3 μg of purified 4L6 IgG was run on 4-12% bris-tris gels on duplicate gels. 4L6 antibody produced in wild-type HEK293T cells was loaded in the first lane of each gel as a control. After transfer, one membrane was probed with anti-rhesus-IgG-HRP and the other membrane was probed with AAL-HRP lectin to visualize the presence of al-6 fucose (FIG. 3B).

Although the IgG probe indicates that equal amounts of 4L6 antibody were loaded per lane, when probed with AAL lectin, varying amounts of al-6 fucose were observed per construct. As expected, constructs 5, 6, and 7 mediated the lowest levels of al-6 fucose. This is consistent with knockdown observed in the real-time PCR and most likely a result of increased spacer lengths as compared to constructs 1 and 2. Levels of al-6 fucose present on 4L6 antibody generated from constructs 6 and 7 were almost undetectable by western blot. These data suggest that knockdown levels would be sufficient to glycoengineer cells in vivo and for the majority of secreted antibody to lack al-6 fucose.

### Example 4: GE-AAV Stable Cell Line Generation

In the experiments from FIG. 3B, it was observed that FUT8 knockdown required around 4 days of construct expression before sufficient levels of knockdown were achieved to produce antibodies with low levels of al-6 fucose. However, in vivo, following intramuscular injection of AAV, muscle cells will continuously express IgG and the shRNA constructs indefinitely. Due to the high rate of transduction of muscle cells following intramuscular injection of AAV and the long-lived nature of this transduction, this scenario was modeled for study in vitro. Constructs 2, 5, and 6 were chosen to make stable cells lines due to the variety of shRNAs in each construct. Construct 2 was chosen over construct 7 due to AAV packaging concerns. Because construct 7 was at the packaging limit, the limited improvement in knockdown was not worth the potential difficulty in AAV packaging.

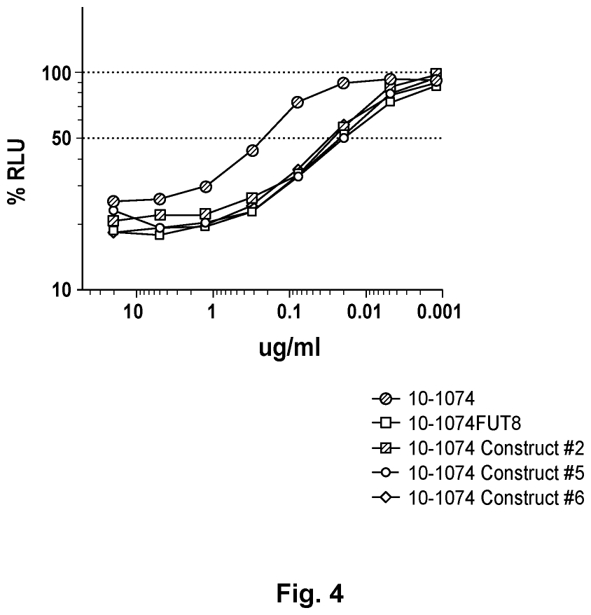

shRNA constructs were cloned into a lentiviral vector with a puromycin resistant selectable marker and a GFP tag. HEK293T cells were incubated with these lentiviruses for 48 hours before adding 1 ug/ml puromycin. Puromycin dosage was escalated to 2 μg/ml after week one and 4 μg/ml after week 2 to select for well-transduced cells. shRNA construct expression was monitored by flow cytometry for GFP expression (FIG. 4A). High levels of GFP were observed in all constructs indicating high shRNA construct expression.

### Example 5: ADCC of Ab 10-1074 Expressed by FUT8 Knockdown Stable Cell Lines

HEK293T cells, FUT8 KO cell lines, and the FUT8 knockdown stable cell lines were transiently transfected with an expression vector for 10-1074 IgG. After 5 days, IgG was purified from the supernatant using protein A columns. ADCC activity was measured by a previously established assay to quantify NK cell activity towards HIV-1 NL4-3 AD8-infected target cells expressing luciferase as previously described.²⁰ Effector cells were combined with infected target cells before addition of 4-fold serial dilutions of purified antibodies. ADCC activity was measured as loss of luciferase activity. The dashed line indicates 50% RLU (relative light units) or 50% ADCC activity against HIV AD8-infected target cells.

As predicted, a >10-fold enhancement of ADCC activity was observed when Ab 10-1074 was generated in the FUT8 KO cell line compared to the wildtype (FIG. 4B). Interestingly, Ab 10-1074 produced in all three FUT8 knockdown stable cell lines also displayed ˜10-fold enhancement in ADCC activity, with only slightly less ADCC activity than observed in the FUT8 KO cell lines. ADCC activity is consistent with minimal amounts of fucose observed on purified 4L6 IgG in FIG. 3B. These data suggest that antibody produced by muscle cells transduced with expression vectors encoding inhibitory RNA in vivo should demonstrate a similar enhancement of ADCC activity.

### Example 6: Fc Mutations in Combination with Glycoengineering

Another common way to increase antibody effector functions, such as ADCC, is through Fc mutations that increase affinity for the Fc receptor. However, little is known about the effects of combining Fc mutations with glycoengineering. In order to determine if there is any added value of combining the two approaches for AAV-vector based delivery of anti-HIV antibodies, common Fc mutations were also explored. The first two Fc mutations tested were the LS (M428L/N434S)²¹ and LALA (L234A, L235A)²² mutations. The LS mutation, while having no effect on ADCC, is known to increase binding to the neonatal Fc receptor and consequently increase serum half-life.²¹ This mutation is commonly used for AAV-delivered antibodies. The second Fc mutation, LALA, is known to abrogate all ADCC activity by disrupting the binding to FcγRIIIA²² These mutants were also tested in combination and annotated LALA-LS (L234A/L235A/M428L/N434S).

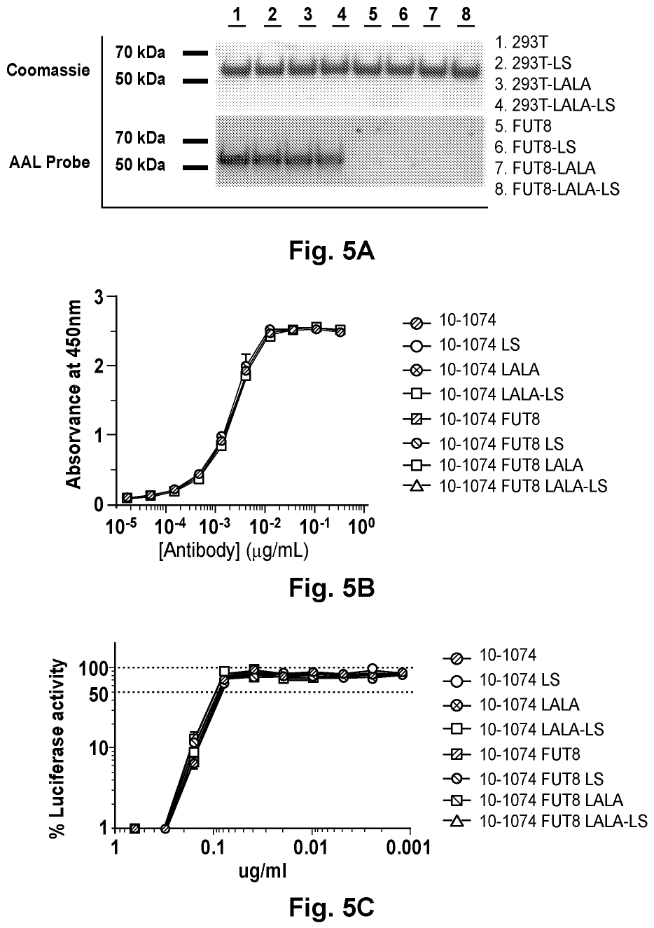

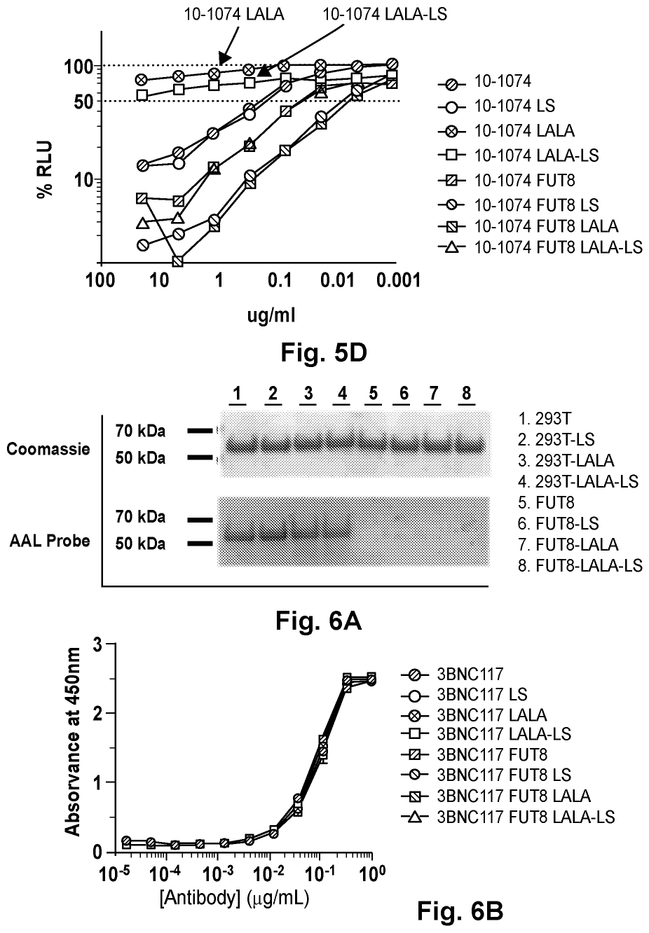

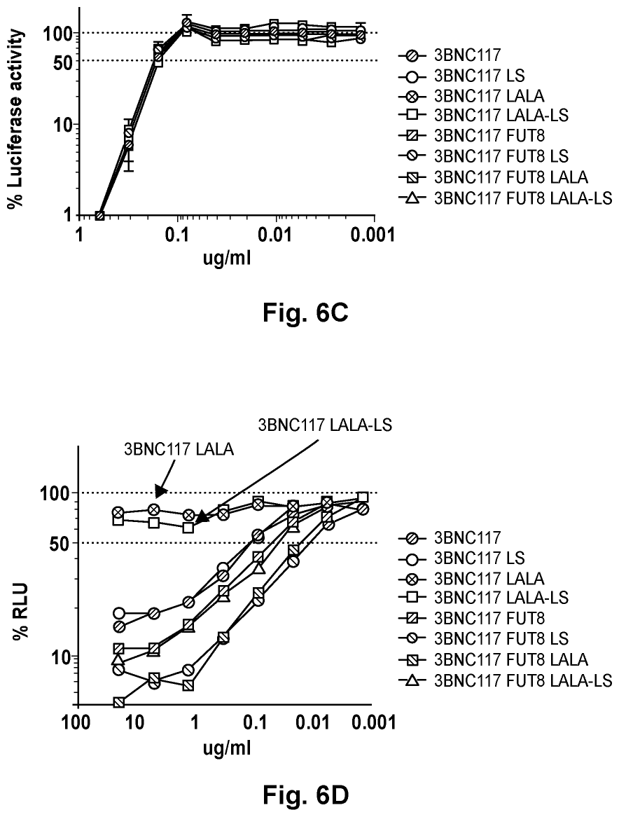

Both Ab 10-1074 and Ab 3BNC117, and their respective Fc mutants, were produced in HEK293T cells and FUT8 KO cells. Antibody was purified and 3 μg of purified IgG was run on duplicate 4-12% bris-tris gels. Ab 10-1074 or Ab 3BNC117 IgG produced in wild-type HEK293T cells was loaded in the first lane of each gel as a control respectively. One membrane was stained as a Coomassie to visualize total protein and the other was transferred to a PVDF membrane and probed with AAL-HRP lectin to visualize the presence of al-6 fucose (FIGS. 5A & 6A). Although equal amounts of antibody were loaded per lane, both Ab 10-1074 and Ab 3BNC117 produced in FUT8 KO cells were devoid of a 1-6 fucose.

Next, the antibodies were analyzed for their ability to bind gp140 using an SF162 gp140 trimer binding ELISA. Starting at an antibody concentration of 1 μg/ml followed by 3-fold serial dilutions, Ab 10-1074 and Ab 3BNC117 variants were serially incubated with 1 μg/ml plate bound SF162 gp140 trimer. High absorbance indicates high binding. As expected, neither glycoengineering nor Fc mutations had any impact on gp140 binding (FIGS. 5B & 6B). Also important to note, the consistency of the assay when comparing different antibodies suggests little to no variability in protein quantification of the IgG. This suggests that any differences observed in ADCC activity are truly due to the antibody modifications and not sample to sample variability.

To determine if glycoengineering in combination with Fc receptor mutations had any impact on neutralization capacity, neutralization assays were performed with Ab 10-1074 and Ab 3BNC117 variants (FIGS. 5C & 6C). Neutralization assays were carried out with HIV-1 NL4-3 AD8. Antibody concentrations started at 1 μg/ml followed by 2-fold serial dilutions. The dashed line indicates 50% RLU (relative light units) representing 50% neutralization activity against AD8. Lowest RLU indicates highest neutralization. As expected, neither glycoengineering nor Fc receptor mutations, individually or in combination, impacted the ability of Ab 10-1074 and Ab 3BNC117 to neutralize AD8.

Ab 10-0174 and Ab 3BNC117 variants were also tested for ADCC activity (FIGS. 5C & 6C). ADCC activity was measured by a previously established assay to quantify NK cell activity towards virus-infected target cells expressing luciferase as previously described.²⁰ Effector cells were combined with infected target cells before addition of 4-fold serial dilutions of purified antibody. The dashed line indicates 50% RLU (relative light units) or 50% ADCC activity against the HIV AD8-infected target cells. The loss of RLU indicates the loss of virus-infected cells during the 8-hour incubation period and represents high ADCC activity.

Consistent with what was observed with 4L6, 10-1074 and 3BNC117 antibodies both display >10-fold enhancement of ADCC when produced in the FUT8 KO cell line compared to wildtype HEK293T cells (FIGS. 5C & 6C). As expected, the LS mutation did not impact ADCC activity even in combination with FUT8 KO. What was most interesting was the LALA mutation. The LALA mutation is known to abrogate ADCC activity. This can be observed in both 10-1074-LALA and 3BNC117-LALA. ADCC activity for the LALA mutants is almost non-existent even at the highest antibody concentrations. However, when 10-1074-LALA and 3BNC117-LALA IgG were produced in FUT8 KO cells, levels of ADCC activity were enhanced to levels greater than wildtype 10-1074 and 3BNC117. Both antibodies exhibited a ˜5-fold enhancement of ADCC activity when the LALA mutant IgG is devoid of al-6 fucose (FIGS. 5C & 6C).

### Example 7: Fc Mutations in Combination with Glycoengineering can Enhance ADCC Activity

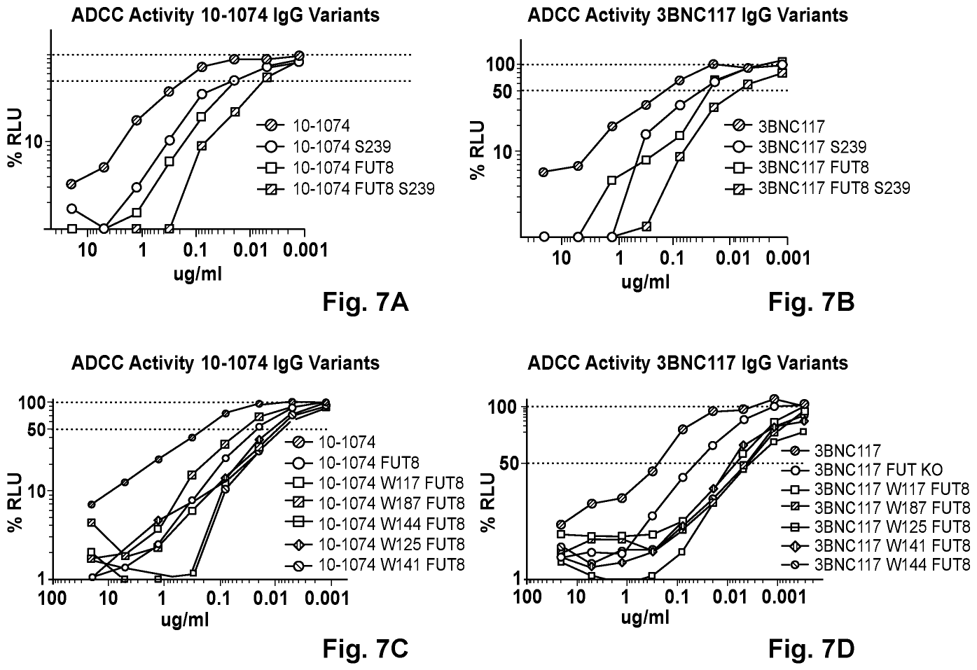

Due to the observation that the ADCC activity of LALA mutant IgG can not only be restored but enhanced by the removal of al-6 fucose, the effect of removal of al-6 fucose on ADCC levels associated with other Fc mutations was examined. For these experiments, the S239 (DFL) mutation (S239D/I332F/A330L) was utilized²³. Both Ab 10-1074-S239 and Ab 3BNC117-S239 were cloned and produced in HEK293T and FUT8 KO cells. ADCC activity was determined from the purified IgG (FIGS. 7A & 7B). Individually, both FUT8 KO and the S239 mutation enhanced ADCC activity by ˜10 fold. However, when combined, 10-1074 FUT8 S239 and 3BNC117 FUT8 S239 displayed an additive effect on ADCC activity, with a 40-60 fold enhancement when compared to the wildtype antibodies.

Asymmetrical Fc mutations were also tested in which one heavy chain has a different set of mutations than the second. Mutants W117, W125, W141, W144, and W187 mutants were all produced in FUT8 KO cell lines. With the exception of 10-1074 W187, all tested mutants displayed enhanced ADCC activity when compared to wildtype 10-1074 and 3BNC117, as well as 10-1074 and 3BNC117 produced in FUT8 KO cells (FIGS. 7C & 7D). These enhancements ranged from 20-80 fold higher ADCC when compared to wildtype IgG. These data suggest that Fc mutations and novel strategies such as asymmetrical Fc mutations can be combined with our glycoengineered-AAV vectors to greatly enhance ADCC activity.

### REFERENCES

1 Fuchs, S. P. et al. AAV-Delivered Antibody Mediates Significant Protective Effects against SIVmac239 Challenge in the Absence of Neutralizing Activity. PLoS pathogens 11, e1005090, doi:10.1371/journal.ppat.1005090 (2015).
2 Martinez-Navio, J. M. et al. Adeno-Associated Virus Delivery of Anti-HIV Monoclonal Antibodies Can Drive Long-Term Virologic Suppression. Immunity 50, 567-575.e565, doi:10.1016/j.immuni.2019.02.005 (2019).
3 Ayyar, B. V., Arora, S. & O'Kennedy, R. Coming-of-Age of Antibodies in Cancer Therapeutics. Trends in pharmacological sciences 37, 1009-1028, doi:10.1016/j.tips.2016.09.005 (2016).
4 Pescovitz, M. D. Rituximab, an anti-cd20 monoclonal antibody: history and mechanism of action. American journal of transplantation: official journal of the American Society of Transplantation and the American Society of Transplant Surgeons 6, 859-866, doi:10.1111/j.1600-6143.2006.01288.x (2006).
5 Scott, A. M., Allison, J. P. & Wolchok, J. D. Monoclonal antibodies in cancer therapy. Cancer immunity 12, 14 (2012).
6 Nielsen, D. L., Andersson, M. & Kamby, C. HER2-targeted therapy in breast cancer. Monoclonal antibodies and tyrosine kinase inhibitors. Cancer treatment reviews 35, 121-136, doi:10.1016/j.ctrv.2008.09.003 (2009).
7 Thomann, M. et al. In vitro glycoengineering of IgG1 and its effect on Fc receptor binding and ADCC activity. PLoS One 10, e0134949, doi:10.1371/journal.pone.0134949 (2015).
8 Yamane-Ohnuki, N. & Satoh, M. Production of therapeutic antibodies with controlled fucosylation. MAbs 1, 230-236 (2009).
9 Peipp, M. et al. Antibody fucosylation differentially impacts cytotoxicity mediated by NK and PMN effector cells. Blood 112, 2390-2399, doi:10.1182/blood-2008-03-144600 (2008).
10 Iannello, A. & Ahmad, A. Role of antibody-dependent cell-mediated cytotoxicity in the efficacy of therapeutic anti-cancer monoclonal antibodies. Cancer metastasis reviews 24, 487-499, doi:10.1007/s10555-005-6192-2 (2005).
11 Iida, S. et al. Two mechanisms of the enhanced antibody-dependent cellular cytotoxicity (ADCC) efficacy of non-fucosylated therapeutic antibodies in human blood. BMC Cancer 9, 58, doi:10.1186/1471-2407-9-58 (2009).
12 Pereira, N. A., Chan, K. F., Lin, P. C. & Song, Z. The “less-is-more” in therapeutic antibodies: Afucosylated anti-cancer antibodies with enhanced antibody-dependent cellular cytotoxicity. mAbs 10, 693-711, doi:10.1080/19420862.2018.1466767 (2018).
13 Goede, V. et al. Obinutuzumab plus chlorambucil in patients with CLL and coexisting conditions. The New England journal of medicine 370, 1101-1110, doi:10.1056/NEJMoa1313984 (2014).
14 Duvic, M. et al. Phase 1/2 study of mogamulizumab, a defucosylated anti-CCR4 antibody, in previously treated patients with cutaneous T-cell lymphoma. Blood 125, 1883-1889, doi:10.1182/blood-2014-09-600924 (2015).
15 FitzGerald, J. M. et al. Benralizumab, an anti-interleukin-5 receptor alpha monoclonal antibody, as add-on treatment for patients with severe, uncontrolled, eosinophilic asthma (CALIMA): a randomised, double-blind, placebo-controlled phase 3 trial. Lancet (London, England) 388, 2128-2141, doi:10.1016/s0140-6736(16)31322-8 (2016).
16 Wren, L. H. et al. Specific antibody-dependent cellular cytotoxicity responses associated with slow progression of HIV infection. Immunology 138, 116-123, doi:10.1111/imm.12016 (2013).
17 Baum, L. L. et al. HIV-1 gp120-specific antibody-dependent cell-mediated cytotoxicity correlates with rate of disease progression. Journal of immunology (Baltimore, Md.: 1950) 157, 2168-2173 (1996).
18 Lambotte, O. et al. Heterogeneous neutralizing antibody and antibody-dependent cell cytotoxicity responses in HIV-1 elite controllers. AIDS (London, England) 23, 897-906, doi:10.1097/QAD.0b013e328329f97d (2009).
19 Moldt, B. et al. A nonfucosylated variant of the anti-HIV-1 monoclonal antibody b12 has enhanced FcgammaRIIIa-mediated antiviral activity in vitro but does not improve protection against mucosal SHIV challenge in macaques. J Virol 86, 6189-6196, doi:10.1128/JVI.00491-12 (2012).
20 Alpert, M. D. et al. A novel assay for antibody-dependent cell-mediated cytotoxicity against HIV-1- or SIV-infected cells reveals incomplete overlap with antibodies measured by neutralization and binding assays. Journal of virology 86, 12039-12052, doi:10.1128/jvi.01650-12 (2012).
21 Zalevsky, J. et al. Enhanced antibody half-life improves in vivo activity. Nature biotechnology 28, 157-159, doi:10.1038/nbt.1601 (2010).
22 Xu, D. et al. In vitro characterization of five humanized OKT3 effector function variant antibodies. Cellular immunology 200, 16-26, doi:10.1006/cimm.2000.1617 (2000).
23 Lazar, G. A. et al. Engineered antibody Fc variants with enhanced effector function. Proceedings of the National Academy of Sciences of the United States of America 103, 4005-4010, doi:10.1073/pnas0.0508123103 (2006).

1. An expression vector comprising a nucleic acid sequence encoding (1) the heavy and/or light chain of an antibody and (2) one or more shRNA sequences targeting fucosyltransferase-8 (FUT8).

2. The expression vector of claim 1, wherein the expression vector encodes both the heavy chain and the light chain of an antibody.

3. The expression vector of claim 1, wherein the expression vector is an adeno-associated viral (AAV) vector.

4. The expression vector of claim 1, wherein the heavy chain comprises one or more mutations in the Fc region which enhances antibody-dependent cell cytotoxicity.

5. The expression vector of claim 4, wherein mutation is an LS mutation (M428L/N434S), a LALA mutation (L234A, L235A), a S239 (DFL) mutation (S239D/1332F/A330L) a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W, K392D/K409D/A330M/K334V, or E356K/D399K/K290Y/Y296W.

6. A composition comprising (a) an expression vector comprising a nucleic acid sequence encoding a heavy chain of an antibody and (b) an expression vector comprising a nucleic acid sequence encoding a light chain of an antibody, wherein (a), (b), or (a) and (b) further comprises one or more shRNA sequences targeting fucosyltransferase-8 (FUT8).

7. The composition of claim 6, wherein the expression vector is an adeno-associated viral (AAV) vector.

8. The composition of claim 6, wherein the heavy chain comprises one or more mutations in the Fc region which enhances antibody-dependent cell cytotoxicity.

9. The composition of claim 8, wherein mutation(s) is an LS mutation (M428L/N434S), a LALA mutation (L234A, L235A), a S239 (DFL) mutation (S239D/1332F/A330L) a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W), K392D/K409D/A330M/K334V, or E356K/D399K/K290Y/Y296W.

10. A method of producing an antibody in vivo, the method comprising delivering to a subject the expression vector of claim 1.

11. The method of claim 10, wherein the heavy chain of the expression vector comprises one or more mutations in the Fc region which enhances antibody-dependent cell cytotoxicity.

12. The method of claim 11, wherein the mutation(s) in the Fc region is an LS mutation (M428L/N434S), a LALA mutation (L234A, L235A), a S239 (DFL) mutation (S239D/1332F/A330L) a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W), K392D/K409D/A330M/K334V, or E356K/D399K/K290Y/Y296W.

13. (canceled)

14. (canceled)

15. (canceled)

16. A method of producing an antibody in vivo, the method comprising delivering to a subject (a) a nucleic acid comprising a nucleic acid sequence encoding a heavy chain of an antibody, (b) a nucleic acid comprising a nucleic acid sequence encoding a light chain of an antibody, and (c) an inhibitory RNA targeting fucosyltransferase-8 (FUT8).

17. The method of claim 16, wherein (a), (b), and (c) are independently present on the same or different expression vectors.

18. The method of claim 16, wherein the expression vectors are AAV vectors.

19. The method of claim 16, wherein the heavy chain comprises one or more mutations in the Fc region which enhances antibody-dependent cell cytotoxicity.

20. The method of claim 19, wherein mutation(s) is an LS mutation (M428L/N434S), a LALA mutation (L234A, L235A), a S239 (DFL) mutation (S239D/1332F/A330L) a C6A-74 mutation (V259I/N315D/N434Y), a HN mutation (H433K/N434F), K392D/K409D/A330M/K334V, E356K/D399K/L234Y/Y296W, K392D/K409D/S239D/A330M/K334V, E356K/D399K/L234Y/K290Y/Y296W, K392D/K409D/A330M/K334V, E356K/D399K/L234Y/K290Y/Y296W, K392D/K409D/A330F/K334V, E356K/D399K/L234Y/K290Y/Y296W), K392D/K409D/A330M/K334V, or E356K/D399K/K290Y/Y296W.

21. The method of claim 16, wherein the inhibitory RNA is shRNA.

22. The method of claim 21, wherein the shRNA comprises the nucleic acid sequence of any one of SEQ ID NOs: 3-7.

23. The method of claim 21, comprising delivering to the subject a composition comprising multiple shRNAs having two or more of SEQ ID NOs: 3-7.
